(19) United States
(12) Patent Application Publication
(10) Pub. No.: US 2019/0374303 A1
(43) Pub. Date: Dec. 12, 2019

(54) FUNCTION MARKER ELEMENT AND METHOD FOR PRODUCTION THEREOF

(71) Applicant: CORTRONIK GMBH, Rostock-Warnemuende (DE)

(72) Inventors: ULLRICH BAYER, BAD DOBERAN (DE); BJOERN RUETER, OSTSEEBAD NIENHAGEN (DE); UWE KNOP, ROSTOCK (DE); GESA BERNGRUBER, ROSTOCK (DE); NIELS GRABOW, ROSTOCK (DE); SABINE ILLNER, ROSTOCK (DE); THOMAS EICKNER, ROSTOCK (DE); KLAUS-PETER SCHMITZ, ROSTOCK (DE)

(21) Appl. No.: 16/429,677

(22) Filed: Jun. 3, 2019

(30) Foreign Application Priority Data
Jun. 11, 2018 (DE) 10 2018 113 810.5

(51) Int. Cl. A61B 90/00; A61L 27/04; A61L 31/02; A61L 31/14; A61L 31/18; A61F 2/82

(52) U.S. Cl. CPC A61B 90/39; A61L 27/047; A61L 31/022; A61B 2090/3966; A61L 31/18; A61F 2/82; A61F 2250/0098; A61L 31/146

(57) ABSTRACT

A marker element for an implant is made from a planar or hollow body-shaped semi-finished product. The semi-finished product is subjected to a plasma-electrolytic treatment on one side, so that a marker element with a surface that is porous on one side is produced.

### CROSS-REFERENCE TO RELATED APPLICATION

This application claims the priority, under 35 U.S.C. § 119, of German patent application DE 10 2018 113 810.5, filed Jun. 11, 2018; the prior application is herewith incorporated by reference in its entirety.

### BACKGROUND OF THE INVENTION

### Field of the Invention

The present invention relates to a marker element for an implant and to a framework for an implant comprising a marker element, and to a method for producing a marker element of this kind and a framework of this kind.

The present invention additionally relates to an implant, in particular an intraluminal endoprosthesis, comprising a framework (scaffold) and comprising a marker element secured to the framework, which marker element, at least in part of its volume, has a different material composition compared to the material of the framework, which material composition preferably comprises radiopaque and/or radiodense material, and to a method for producing an implant of this kind.

The implants are endovascular prostheses (endoprostheses, stents) or other implants which can be used for the treatment of stenoses (vasoconstrictions). They usually have a framework in the form of a hollow-cylindrical or tubular basic mesh, which is open at both longitudinal ends of the tube. A framework of this kind usually has multiple interconnected bars (struts), which form the basic mesh. The tubular basic mesh of an endoprosthesis of this kind is inserted into the vessel to be treated and is used to support the vessel. Further framework forms are likewise possible. The present invention also relates to implants which can be used in the field of orthopaedics, for example for the skull region, and in particular implants which have a low radiopaque visibility on account of their small size and wall thickness. The invention can also be used for stents in the neurovascular field. Here, the focus is on using absorbable Mg stents to hold open the blood vessels that supply the brain. These systems are used in the field of prevention of acute ischaemic strokes.

Stents or other implants often comprise metallic materials in their framework. Here, the metallic materials can form a biodegradable material, wherein polymer biodegradable materials can also be contained.

The term “biodegradation” is understood to mean hydrolytic, enzymatic and other metabolic degradation processes in a living organism which are caused primarily by the bodily liquids coming into contact with the endoprosthesis and leading to a gradual dissolution of at least large parts of the framework or of the implant. The term “biocorrosion” is often also used for the biological degradation process in the case of implants made of metals. The term “bioresorption” comprises the subsequent resorption of the degradation products by the living organism. The objective of using biodegradable implants lies in the fact that they are broken down by the organism at a time when they are no longer required, for example in respect of their supporting effect, and consequently are not provided any longer than necessary in the form of a foreign body in the organism.

Materials (basic material) suitable for the framework of biodegradable implants can consist of one material or a plurality of materials. Examples of suitable polymer compounds are polymers from the group of cellulose, collagen, albumin, casein, polysaccharides (PSAC), polylactide (PLA), poly-L-lactide (PLLA), polyglycolic acid (PGA), poly-D,L-lactide-co-glycolide (PDLLA-co-GA), polyhydroxybutyric acid (PHB), polyhydroxy valeric acid (PHV), poly(alkyl)carbonates, polyorthoesters, polyethyelene terephthalate (PET), polymalonic acid (PML), polyanhydrides, polyphosphazenes, polyamino acids and copolymers thereof, and hyaluronic acid. The polymers can be present in pure form, in derivatized form, in the form of blends, or as copolymers, depending on the desired properties. Metallic biodegradable materials are based on alloys of magnesium, iron, zinc and/or tungsten.

The position of a stent or other implants is determined often by means of imaging methods, for example by means of an x-ray device. Due to the low atomic number and the low density of the biodegradable material, for example magnesium and alloys thereof, the radiopaque visibility of the medical implants produced therefrom is very low. In order to overcome this disadvantage, it is known to provide medical devices with marker elements which, at least in part of their volume, have a different material composition as compared to the material of the framework. These so-called (x-ray) markers or marker elements contain in particular a material that absorbs the x-rays and/or other electromagnetic rays more strongly (referred to hereinafter as radiodense or radiopaque material) than the material of the framework or the bodily environment of the patient. As a result they are visible relative to their surroundings. Based on the determined position of the marker elements on the framework, there usually being several such marker elements, the position and angular position of the implant relative to the surrounding organs can be determined. With use of a biodegradable framework a non-resorbable radiopaque or radiodense material (for example Ta, Au, W) is often used for reasons of sufficient radiopaque visibility.

A marker element of this kind is often trimmed or cut out from a semi-finished product consisting of the material of the marker element and is integrated in an implant in such a way that it is glued into a corresponding opening (eyelet), which for this purpose is provided on the framework of the implant (for example at both ends in the axial direction of the implant). Marker elements and implants of this kind are known for example from published patent application US 2011/0319982 A1 and its European counterpart patent EP 2 399 619 B1 and from US2017/0119555 A1 and its European counterpart EP 3 165 238 A1.

The technical requirement on implants with integrated marker elements is that the marker elements remain in the framework composite with a sufficient force of adhesion over a long period of time. This was achieved previously exclusively by technological means that may be integrally bonded, positively engaged or frictionally engaged in relation to the framework or the opening of the framework into which the marker element is introduced. In the event of an inadequate connection of the marker element to the framework of the implant, an undesirable local element may form or contact corrosion may be produced by means of a metallic contact between marker element and framework material. This would lead to a premature separation of the marker element from the framework of the implant, which would promote an undesirable fragment formation and embolization. In addition, the material of the marker element should generally be prevented from influencing the degradation of the framework.

### SUMMARY OF THE INVENTION

It is an object of the present invention to create an implant or a framework for an implant in which a local element formation and corrosion are avoided or at least reduced. Accordingly, a marker element which, after integration into the implant, does not form a local element and which prevents corrosion should be created. Furthermore, economical, simple and easily automated methods for producing a marker element of this kind, a framework with an integrated marker element of this kind, or an implant with a framework of this kind shall be described.

With the above and other objects in view there is provided, in accordance with the invention, a method for producing a marker element for an implant, the method comprising:
providing a planar or hollow body-shaped semi-finished product consisting of a material of the marker element;
cutting out at least one portion from the semi-finished product, in such a way that the portion remains connected via at least one web to a remaining material of the semi-finished product;
with the portion still connected to the semi-finished product via the at least one web:
   covering a first surface of the portion; and
   subsequently performing plasma electrolytic treatment of a second surface of the portion that is different from the first surface, and thereby forming a porous layer at least on the second surface of the portion by the plasma electrolytic treatment; and
separating the portion by severing the at least one web, wherein the separated portion of the semi-finished product forms the marker element.

In other words, the method according to the invention for producing a marker element for an implant from a planar or hollow body-shaped semi-finished product consisting of the material of the marker element has in particular the following steps: cutting out at least a portion from the semi-finished product, in such a way that the portion is connected via at least one web to the rest of the material of the semi-finished product, while the portion is still connected to the semi-finished product via the at least one web: covering a first surface of the portion and performing a subsequent plasma electrolytic treatment of at least a second surface of the portion, wherein the at least one second surface is different from the first surface, wherein a porous layer is formed at least on the second surface of the portion by the plasma electrolytic treatment, and

separating the portion by severing the at least one web, wherein the separated portion of the semi-finished product forms the marker element.

The method according to the invention produces a topographical structuring of the second surface of the marker element, which serves as an abluminal side of the marker element following the integration into the framework of an implant, with the option of acceleration of the ingrowth of the marker element into the tissue.

By means of the plasma electrolytic oxidation, a high surface porosity is produced on the second surface, and in an exemplary embodiment also partially at least one side face of the marker element, which runs transversely to the second surface, and this can be used to attain a high wetting grade for coatings to be applied subsequently.

The planar or hollow body-shaped semi-finished product is formed for example by a plate, a hollow cylinder (tube) or a three-sided, four-sided or more than four-sided, preferably straight hollow prism, which is open at both ends in the direction of a longitudinal axis.

The semi-finished product, and accordingly the marker element produced from the semi-finished product, consists of a metal or metal alloy containing at least one metal from the group comprising the elements tungsten, tantalum, gold and platinum. The semi-finished product, and accordingly the marker element, particularly preferably consists of tantalum or a tantalum alloy. The specified materials of the marker element have good x-ray absorption properties and can be provided very easily on their surface with a dense, passivating and insulating (i.e. electrically non-conducting) oxide layer by means of the plasma electrolytic oxidation. They hereby allow a marker element to be formed with minimal size. The semi-finished product is preferably produced by drawing from the material in question.

The at least one portion, preferably a plurality of portions arranged adjacently along the entire width of the plate or the entire or partial periphery of the hollow cylinder or hollow prism, is cut out from the semi-finished product in such a way that each portion is still connected to the rest of the material of the semi-finished product via at least one web. Each portion is preferably connected to the rest of the material of the semi-finished product via at least two webs, which are opposite one another along the periphery of the portion. Each web forms a predetermined breaking point for the separation of a corresponding portion from the semi-finished product. Each portion is cut out from the semi-finished product preferably by means of laser, in such a way that the portion in question is freed along its periphery from the semi-finished product (apart from the web(s)), i.e. a peripheral, continuous incision separates the material of the portion in question (apart from the web(s)) from the rest of the material of the semi-finished product. Each portion (apart from the web(s)) already has the form of the marker element produced herefrom, for example a disc form. The disc can have a circular, elliptical, triangular, quadrangular or other polygonal or rounded basic form, which is formed in each case by the bottom and top faces. In particular, each portion has a first surface, which forms the bottom face of the marker element, a second surface, which is opposite the first surface and which forms the top face of the marker element, and at least one side face, which connects the first and the second surface to one another. The height of the side face corresponds to the thickness of the portion of the marker element produced herefrom, wherein the plasma electrolytically generated coating and any further coatings must be taken into consideration when determining the thickness of the marker element. Furthermore, the height of the side face also corresponds to the thickness of the semi-finished product plate or the wall thickness of the hollow cylinder or of the hollow prism. The at least one side face corresponds to a certain extent to the cutting-edge of the portion cut out.

By means of the plasma electrolytic treatment a porous, dielectric surface is created at the uncovered areas, in particular the second surface and possibly the at least one side face, which optionally can be further modified in additional immersion steps (see below). For a semi-finished product consisting of tantalum or a tantalum alloy, the natural crystalline oxide layer already provided obtains, by means of this process, an amorphous oxide layer with a layer thickness between 0.3 μm and 10 μm, preferably between 0.5 μm and 4 μm. Tantalum markers, at the end of the plasma electrolytic treatment, have a pore structure with a mean pore diameter between 0.1 μm and 2 μm, and the mean pore depth is slightly smaller than the layer thickness. Here, the pore diameter can be determined by scanning electron microscopy (SEM) or confocal laser scanning microscopy (CLSM). The pore depth in the metallographic cross-section is determined likewise by means of the aforementioned methods.

The first surface of the portion, with use of a semi-finished product in the form of a hollow cylinder or hollow prism, can be covered for example by introducing an elongate (approximately cylindrical) polymer balloon or a thermally supercooled tube made for example of silicone or a tube of variable diameter with separate inner mandrel into the interior volume of the hollow cylinder or hollow prism. By means of pressure application, the balloon presses against the inner surface of the semi-finished product and thus seals the inner surface (inclusive of the first (inner) surface of the at least one cut-out portion for the marker element) with respect to subsequently acting external medium infiltration forces. At the covered first (inner) surface of the at least one cut-out portion of the semi-finished product, there is thus no change to the surface as a result of the plasma electrolytic treatment.

For the plasma electrolytic treatment, the semi-finished product is electrically contacted and coated with the aid of the plasma electrolytic process, which is performed in a specific acid mixture at high bath voltages. On account of the covering of the first (inner) surface of the cut-out portion, no coating is provided there, but instead only at the exposed surfaces, i.e at the uncovered second surface and possibly at least at one side face of the at least one cut-out portion. The second surface is opposite the first surface and is preferably parallel thereto.

During the plasma electrolytic treatment (coating), a pulsed voltage is applied to the semi-finished product by means of anodic contacting. Via the at least one web, the at least one cut-out portion is also in electrically conductive contact with the voltage source. The amplitude of the voltage, for part of the treatment time, exceeds a bath voltage characteristic for the material of the main body and preferably rises during the course of the treatment. A voltage between 450 V and 1,000 V, preferably between 500 V and 600 V, is advantageously applied. The contacting material consists of a suitable acid-resistant metal, for example of a titanium wire. The semi-finished product is now immersed in an acid-containing solution. Following application of a pulsed, continuously rising bath voltage, which is characterised by long pulse pauses (pulse off), the material of the semi-finished product with the at least one cut-out portion is oxidized. Here, the pulse pauses (pulse off) are at least as long as the pulse durations (pulse on), but preferably one-and-a-half times as long. In an embodiment of the invention pulse durations in a range between 30 μs and 600 μs, preferably between 100 μs and 300 μs, particularly preferably 200 μs, and pulse pauses in a range of from 175 μs to 600 μs, preferably between 400 μs and 550 μs, particularly preferably 450 μs, are expedient.

The plasma electrolytic treatment is preferably carried out with a high maximum applied voltage, preferably with a maximum applied voltage of more than 180 V with a pulsed voltage source. The porosity of the oxide layer is brought about by the high-voltage.

Specific acid mixtures are preferred, for example concentrated phosphoric acid (85%) or concentrated phosphoric acid (85%) with concentrated sulphuric acid (preferably in the ratio: 90:10, v/v). A rectangle or trapezium profile is expedient as voltage profile.

Pulse parameters:
the pulse on and pulse off times correlate with one another
a process window can be spanned between the following corner points (in each case pulse on to pulse off time):
   30 μs to 175 μs, 30 μs to 600 μs, 600 μs to 500 μs, 600 μs to 600 μs
the optimal pulse parameters lie at 200 μs pulse on to 450 μs pulse off.

A plasma electrolytic treatment of this kind is preferably carried out for the duration of from 1 to 3 min.

The semi-finished product is preferably rinsed in demineralised water following the plasma electrolytic treatment.

The at least one portion is separated from the semi-finished product after the plasma electrolytic treatment by cutting (for example by means of laser) or breaking the at least one web by means of which the portion in question is connected to the semi-finished product. As applicable, the covering (for example the balloon) is removed from the portion of the semi-finished product prior to the separation of the at least one portion. The marker element thus created can then be inserted into an opening (eyelet) of the mesh and adhesively bonded thereto. Further treatments and/or coatings (see below) of the portion are performed as appropriate prior to the separation of the at least one portion.

The advantage of the porous layer produced during the course of the method by means of plasma electrolytic treatment on the second surface and possibly at least one side face (of the cutting-edge) lies in particular in the oxidic basic structure, which leads to dielectric, i.e. insulating, surface properties and therefore, following the integration into the framework of an implant, prevents metal/metal contact towards the opening (eyelet) and therefore the framework. The oxidic basic structure is additionally mechanically extremely robust and can plastically deform without causing damage to the layer. This opens up the possibility of an automation of the processes for introducing the marker elements into the framework (mounting) without the risk of particle generation. In other words, the creation of a robust, resistant porous layer in particular at the at least one side face by the method according to the invention promotes a mechanical grip in the event of an automated mounting of the marker elements in the framework.

In a development of the invention, prior to the covering of the first surface of the portion and prior to the plasma electrolytic treatment, the semi-finished product is subjected in the region of the at least one cut-out portion to a pickling treatment with hot caustic potash or another hydrofluoric acid-containing acid mixture, for example by means of a solution formed from HNO₃ and HF. To this end the semi-finished product, and together therewith the at least one cut-out portion, is immersed in the acid. Here, (laser) cutting burrs are removed and modifications are performed on all surfaces of the portion.

Since the first surface of the cut-out portion or the bottom face of the marker element is sealed off during the plasma electrolytic treatment, this surface is not altered by the plasma electrolytic treatment. In this exemplary embodiment, following the finishing of the marker element, said surface therefore has the structure created by the pickling treatment. The marker element, at the time of integration into the framework of the implant, is arranged such that the first surface or the bottom face of the marker element lies on the luminal side. The resultant luminal marker element inner surface consists of the groove-like (furrow-like) structure oriented at 0° relative to the framework axis and created by the pickling process, which structure is created by particles of the marker element volume oriented in a preferred direction and is caused by the drawing of the semi-finished product. This groove-like structure counteracts an adhesion of the blood platelets and thus reduces the risk of clotting, preferably also at a time following the dissolution of a PLLA layer arranged optionally thereon.

The semi-finished product can then be rinsed with the at least one cut-out portion by means of demineralised water.

In a further exemplary embodiment, in the region of the at least one cut-out portion, the semi-finished product is optionally firstly immersed in a solution and hereby coated following the plasma electrolytic treatment, wherein the solution comprises a crosslinker and at least one material from the group containing proteins, further growth factors, enzymes, antibodies and peptides.

In this exemplary embodiment there is an additional modification of the marker surface with adhesion molecules. The still-covered semi-finished product, for example sealed by the balloon structure, is coated following the plasma electrolytic treatment with bifunctional crosslinkers and then with proteins or peptides. These coating processes are performed by immersing the semi-finished product in an appropriate solution comprising these compounds. The proteins (for example VEGF—vascular endothelial growth factor) and peptides (for example RGD peptides (adhesion peptides)) have the purpose of holding the marker element at the implantation site and of helping it to become ingrown. An unintentional release of the marker element following degradation of the framework and thus a transport of the free marker element through the vessel system can thus be prevented.

Even after coating of the framework inclusive of the marker elements with a polymer active substance coating, it is possible that the proteins or peptides remain active and only after the degradation of the layer lead to an additional fixing in the tissue. In this case the modification with adhesion molecules constitutes a protection mechanism that increases the long-term reliability of the framework.

The method of the protein/peptide coating is formed of two sub-steps, wherein the semi-finished product, for example the tantalum hollow cylinder, is immersed successively in the region of the at least one cut-out portion, preferably with balloon still arranged inside, in the individual coating solutions a), b), specifically
a) a mixture of a crosslinker, with initiator and starter in a suitable solvent.

Glutardialdehyde, 1-ethyl-3-(3-dimethylaminopropyl)carbodiimide/N-hydroxysuccinimide (EDC/NHS), am inopropyltriethoxysilane (APTES) or others can be used as crosslinkers. Here, the reaction on the second surface of the cut-out portion can also be thermally activated.
b) a mixture of proteins, such as VEGF, further growth factors, enzymes or antibodies, or peptides, such as adhesion peptides, (RGD peptides), which all have an affinity for the cells of the vessel wall.

These bind covalently to the activated marker element surface or to the crosslinkers already provided there. Here, the reaction on the marker element surface can also be thermally activated.

The type of coating is selected such that the coating solution wets the marker element surface and the pore structure thereof. In an exemplary embodiment the pores of the porous layer are filled with the protein/peptide coating solution by applying a negative pressure (for example in a desiccator). Either rinsing or drying processes (for example by means of demineralised water) or (after the separation from the semi-finished product) an immediate adhesive bonding in an opening (eyelet) of the framework can then be performed.

In a development of the invention an organic compound and/or an inorganic compound capable of elution are/is immobilised in the pore structure of the porous layer after the plasma electrolytic treatment.

For example, Ca, K and Mg compounds are immobilised as inorganic compounds. These can be physiologically occurring salt compounds, such as phosphates, chlorides or carbonates. Since the porous layer on the second surface (top face) lies in the abluminal side following the integration into the framework or the implant, the inorganic compounds in the event of contact with cell tissue lead to an intensified interaction with the endothelial cells. This results in a quicker incorporation of the marker element into the vessel wall.

Compounds similar to BMPs, which in the field of orthopaedics bring about an acceleration of osteosynthesis and which likewise support the incorporation of the marker element in the vessel wall, can be used as organic compound.

The problem described above is additionally solved by a method for producing a framework comprising a marker element for an implant having the above method steps for producing the marker element and the additional method step in which the portion separated from the semi-finished product and forming the marker element is introduced into an opening (eyelet) of the framework and is glued into the opening. The opening is preferably continuous. Here, as already discussed above, the marker element is glued into the framework in such a way that the first surface of the portion (bottom face of the marker element), which has been created by the pickling process, forms a luminal face and the second surface of the portion (top face of the marker element) with the porous layer forms an abluminal face on the framework or the implant. Due to the luminal, pickled surface of the marker element, the risk of formation of clots is reduced, whereas by means of the porous layer on the abluminal side the ingrowth of the marker element into the tissue is promoted and local element formation with the framework is prevented.

In a further preferred exemplary embodiment the at least one marker element is secured to the framework by an adhesive, preferably by a polymerisable adhesive. It has specifically been found that in particular by means of an integrally bonded connection a simple connection not mechanically stressing the filigree framework is attained. In addition, a form fit can be realised by adapting the shape of the marker element and the shape of the opening (eyelet, receptacle) to the framework of the implant in which the marker element is introduced.

In a preferred embodiment resilient polymer adhesives are used. Suitable resilient adhesives comprise silicones, polylactides, polyhydroxybutyric acid, thermoplastic elastomers (TPE) and blends thereof. Resilient plastics have the advantage that they contribute to an improved trackability, i.e. to the adaptation to the surrounding tissue as the endoprosthesis is advanced during the insertion process, and therefore a premature loss of a marker element is avoided.

Polyurethane for example or a degradable polymer (for example PLLA such as L210 or L214 by Evonik) can be used as advantageous polymer-based adhesive. These adhesives are particularly biocompatible and in addition are good electrical insulators.

As already explained above, it is advantageous if the at least one marker element is secured to the framework, in particular in an opening of the framework, preferably by means of an above-mentioned polymer-based adhesive, since the securing of the marker element to the framework is simple and can be performed in an automated manner.

In a development of the invention the gluing is realised by means of a polymer solution, for example a PLLA/CHCl₃ solution, applied previously to the marker element separated from the semi-finished product, wherein the polymer solution is applied preferably by pressing the marker element manually or in an automated manner, for example with the aid of a punch or vacuum tweezers, into the (possibly slightly hardened) polymer solution and/or wetting the marker element with the polymer solution (for example by spraying) and/or filling the gap between the marker element and opposite edge of the opening of the framework, and the polymer solution is then cured.

In a further exemplary embodiment the framework is provided at least in a predefined region, prior to gluing of the marker element, with a coating containing a pharmaceutically active substance (for example immunosuppressant, cytostatic).

Here, a “pharmaceutically active substance” (or therapeutically active or effective substance) is understood to mean a plant, animal or synthetic active substance (medicament) or a hormone, which is used in a suitable dose as a therapeutic agent for influencing conditions or functions of the body, as a substitute for active substances produced naturally by the human or animal body, such as insulin, and for eliminating or neutralising pathogenic agents, tumours, cancer cells or exogenous substances. Possible active substances of this kind include Everolimus, Sirolimus, Paclitaxel or Heparin, for example.

Alternatively or additionally, the framework is usually provided in a predefined region, following the gluing of the at least one marker element, with a coating containing a pharmaceutically active substance.

The above problem is additionally solved by a disc-shaped marker element for an implant having a bottom face forming a first surface, a top face forming a second surface, and at least one side face connecting the bottom face and the top face, wherein the top face forms an abluminal face once the marker element has been secured to the framework of the implant. In accordance with the invention the top face and possibly also at least one side face of the marker element is covered at least in part by a porous layer, which was generated by plasma electrolytic treatment, as already discussed further above in detail.

In an exemplary embodiment the porous layer has a pore size between 0.1 μm and 2 μm and/or a layer thickness between 0.3 μm and 10 μm, preferably between 0.5 μm and 4 μm.

As already discussed above, the marker element in one exemplary embodiment has a groove-shaped pickled structure on the bottom face if, prior to the plasma electrolytic treatment, a pickling treatment of the at least one portion was performed on the semi-finished product using an acid.

With the above and other objects in view there is also provided, in accordance with the invention, a framework for an implant comprising a marker element as described above, wherein the marker element is glued into an opening (eyelet) of the framework in such a way that the top face of the marker element forms an abluminal face. Accordingly, the bottom face of the marker element, which has the preferably groove-shaped pickled structure, can form an abluminal face.

With the above and other objects in view there is also provided, in accordance with the invention, an implant having a framework, as described.

Other features which are considered as characteristic for the invention are set forth in the appended claims.

Although the invention is illustrated and described herein as embodied in a function marker element and method for the production thereof, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a plan view from above of a portion of a framework of an implant;

FIG. 2 is a plan view from above of a marker element;

FIG. 3A and 3B are images recorded by scanning electron microscopy of the outer side (FIG. 3A) and the inner side (FIG. 3B) of a region of a hollow-cylindrical semi-finished product with multiple portions for marker elements following removal of the slag residues and any impurities, in each case in a view from the side in question;

FIG. 4 illustrates an inner side of the region of the hollow-cylindrical semi-finished product according to FIG. 3B with a portion for a marker element after the pickling step in an image from the side recorded by light microscopy;

FIG. 5A-5C are images recorded by scanning electron microscopy from above and in different magnifications of regions of an abluminal top face of a marker element after the plasma electrolytic treatment in various magnifications, wherein FIG. 5B and 5C each show a scratch created in the surface by mechanically pressing in a sharp object; and

FIG. 6A-6B show a region of the abluminal top face and the side face of the marker element in an oblique image from the side, recorded by scanning electron microscopy, in different magnification.

### DETAILED DESCRIPTION OF THE INVENTION

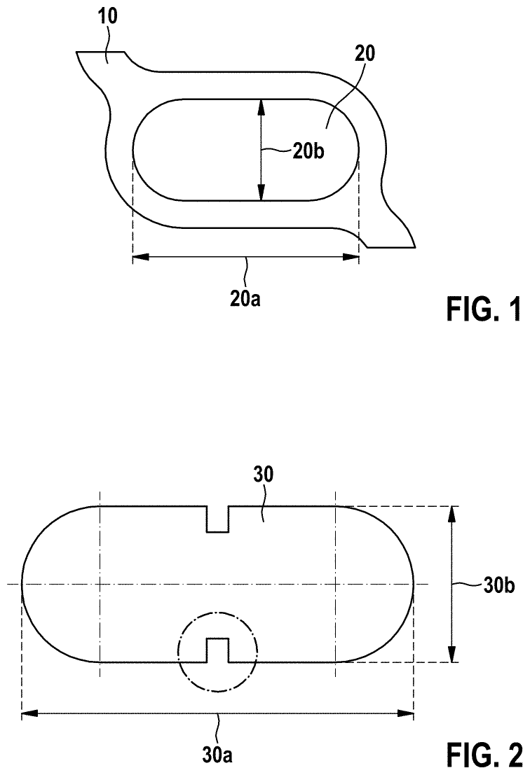

Referring now to the figures of the drawing in detail and first, particularly, to FIG. 1 thereof, there is shown a portion of a framework 10 of an implant according to the invention in the form of a medical stent, for example consisting of the degradable magnesium alloy WE43. The image shows a continuous opening (“eyelet” hereinafter) 20 arranged for example at the distal or proximal end of the framework 10 and having a rounded rectangular shape. One eyelet 20 or three such eyelets 20 offset by 120° is/are preferably provided at the distal and/or at the proximal end of the framework 10 of the implant as part(s) of the framework, for example on a strut. Here, the framework is preferably formed as a hollow-cylindrical mesh with multiple struts. By way of example, the dimensions of the eyelet 20 are 800 μm (dimension 20a in FIG. 1)×350 μm (dimension 20b in FIG. 1).

A radiopaque marker element 30, as illustrated in FIG. 2, can be arranged in the eyelet 20. As described hereinafter, it can be secured to the eyelet 20 by means of an adhesive layer.

The marker element 30 for example is made predominantly of tantalum or a tantalum alloy. The thickness of the marker element 30 is for example 100 μm and corresponds preferably to the wall thickness of the framework 10. By way of example, the dimensions of the marker are 750 μm (dimension 30a in FIG. 2)×300 μm (dimension 30b in FIG. 2).

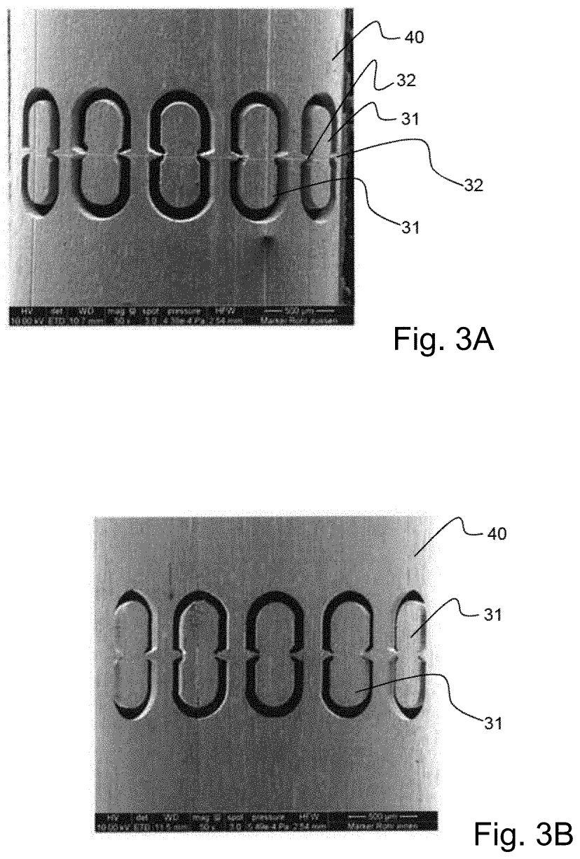

Referring now to FIG. 3, in order to produce the marker element 30, portions 31 with the shape of the marker element 30 are firstly cut out with the aid of a laser from a semi-finished product in the form of a drawn hollow cylinder 40 made of tantalum or a tantalum alloy, for example having an outer diameter of 2.5 mm. Here, each portion 31 is also connected to the hollow cylinder 40 via two webs 32. Slag residues, laser cutting burrs and any impurities on the hollow cylinder 40 with the portions 31 are then removed by pickling with an acid mixture formed for example of HNO₃ and HF. Subsequently, a multi-stage rinsing step in demineralised water is performed. For the pickling the hollow cylinder with the portions 31 is immersed multiple times into the acid mixture. The condition of the outer side and the inner side of a hollow cylinder 40 of this kind with the portions 31 is shown in FIG. 3A and 3B.

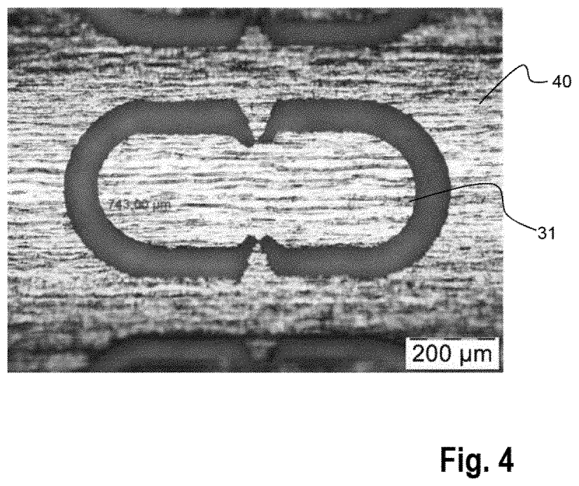

FIG. 4 shows the inner side of the hollow cylinder 40 with a portion 31 in an enlarged view. The inner side of the portions 31, which is also referred to as the first surface (and therefore the bottom face of the marker element 30 produced therefrom) is characterized by a structure, produced by the drawing process of the hollow cylinder 40 and the subsequent pickling, having a plurality of grooves or furrows. These run, following the arrangement of the marker element 30 in the frame 10, parallel to the axis of the frame or parallel to the flow direction of the blood flowing through the stent. The structure counteracts an adhesion of blood platelets and thus reduces the risk of thrombosis (clotting), also, and in particular, at a time following the dissolution of an optional PLLA layer applied to the surface of the framework.

A balloon with a diameter of 2.35 mm (after expansion of the balloon) is then introduced into the hollow cylinder 40. The balloon is expanded by means of pressure application. Following the expansion the balloon lies with a form fit against the inner side of the hollow cylinder 40 and seals it fully. The outer, second surface of the portions 31 of the hollow cylinder 40 and the side faces 33 can thus be subjected to further process steps without the first surface of the portions 31 of the hollow cylinder shown in FIG. 4 coming into contact with further media.

The hollow cylinder with the portions 31 is now subjected to a plasma electrolytic treatment. The oxidation of the chemically stable element tantalum is brought about by the locally limited plasma discharges created at bath voltages>180 V. Here, individual plasma discharges systematically scan the uncovered surface of the portions 31, specifically the outer surface (see FIG. 3A) and at least partially the side surfaces 33 of the portions 31 exposed by the laser cutting.

The plasma electrolytically treated hollow cylinder 40 with the portion 31 is then rinsed multiple times in demineralised water, and the portions 31 are separated from the hollow cylinder 40 by severing (breaking) the webs 32. Each separated portion forms a marker element 30. The rinsing and breaking of the portion 31 are performed here after the removal from the hollow cylinder 40 of the balloon covering the inner side.

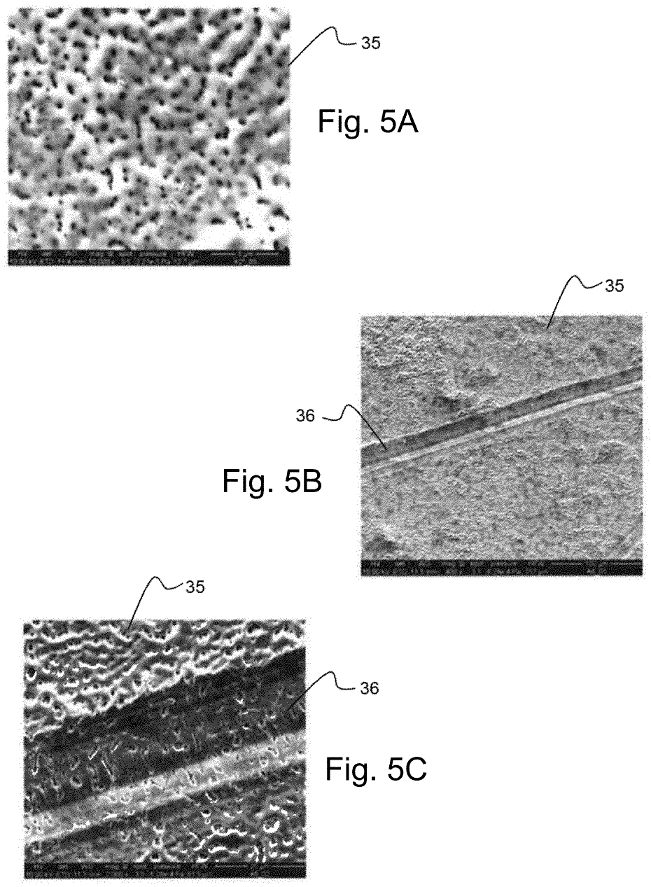

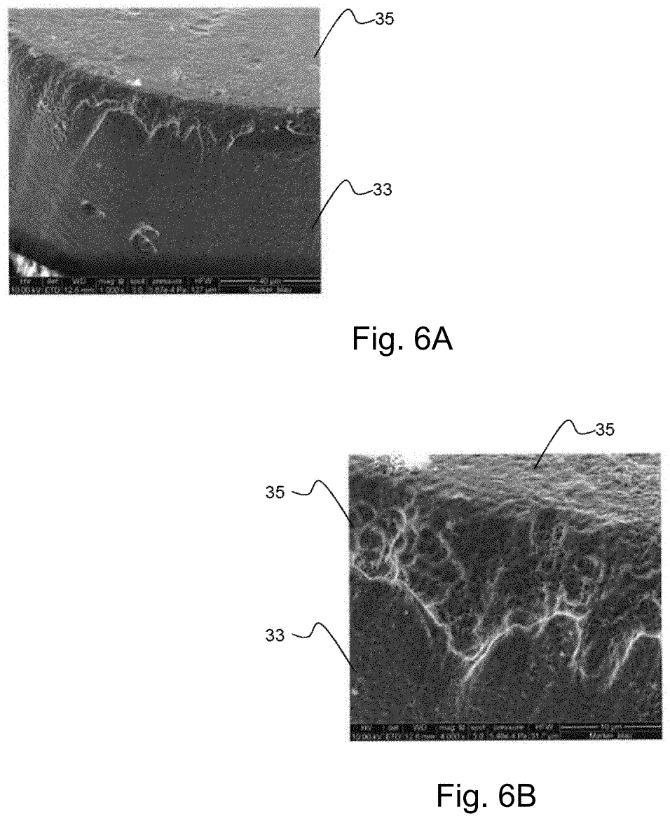

The marker element 30, due to the plasma electrolytic treatment, obtains a porous surface typical for this process, which consists for the main part of Ta₂O₅. The thickness of the porous layer 35 produced by the plasma electrolytic treatment is for example between 0.5 μm and 4 μm. Due to the conversion characteristics of plasma discharges, the original outer geometry of the marker element 30 relative to the portion 31 is maintained. The porous layer 35 generated by the plasma electrolytic treatment is shown in FIG. 5A to 5C. The porous layer 35 has a high adhesive strength on account of the material interconnection to the metallic substrate arranged beneath formed from tantalum or a tantalum alloy. This is evident on the basis of the scratches 36 shown in FIG. 5B and 5C, which are produced by pressing in with a sharp object (indenter). The pore structure of the porous layer 35 stops cracks which are produced by mechanical action.

The surface structuring by the layer 35 also leads to a significant increase of the actual surface of the marker element 30 at least by a factor of 2. This is an essential precondition for a high immobilization capacity of the marker element 30.

For arrangement on a framework 10, the hollow cylinder 40 is removed from the desiccator, installed in a removal frame and immediately gripped mechanically, with a portion 31 being broken out from the hollow cylinder so that the marker element 30 is created. The framework 10 will have been provided with a polymer active substance coating (for example PLLA/Sirolimus) shortly beforehand. Following the positioning of the marker element 30 in the eyelet 20, the marker element is pressed into the solvent-containing coating (for example PLLA in chloroform 2/98), which is still plastically deformable. In addition, the resultant gap between marker element 30 and framework 10 can be filled with the polymer solution. A sufficient holding force of the marker element 30 in the eyelet 21 is then brought about by the evaporation of the solvent from the solution, as is then also a curing of the polymer coating. Thus, neither the biofunctionalized, abluminal marker side nor the luminal marker side, structured in the blood flow direction, is covered by a polymer layer. The framework 10 with the marker element 30 can then be assembled in the catheter system.

Due to the porous layer 35, which is formed by the plasma electrolytic treatment, it is ensured that no metal/metal contact between framework 10 and material of the marker element 30 is created, that local element formation is thus inhibited reliably, and that there is no influencing of the further course of degradation of the framework 10.

In a further, alternative exemplary embodiment the marker element 30 can be immersed in a polymer solution (for example PLLA in chloroform 2/98) prior to the positioning in the eyelet 20. In this embodiment a filling of the gap between marker element 30 and framework 10 is not absolutely necessary. After the positioning of the marker element 30 in the eyelet 20, a sufficient holding force of the marker element 30 in the eyelet 20 is provided by the curing of the polymer coating. The framework 10, inclusive of marker element 30, can then be coated by a polymer active substance layer, and the framework can then be assembled in the catheter system.

In an alternative exemplary embodiment the plasma electrolytically treated hollow cylinder 40 can be rinsed multiple times in demineralised water following the removal of the balloon and then additionally immersed in a solution containing the crosslinker APTES for example (aminopropyltriethoxysilane). By applying a negative pressure in a desiccator, the pores are saturated with the solution. Here, the reaction on the surface of the portions 31 can also be thermally activated. There is then additionally a reaction with adhesion peptides (for example RGD peptides), which have a particular affinity for the cells of the vessel wall. These bind covalently to the surface activated with APTES. The hollow cylinder 40 with the portions 31 is then rinsed/dried and deposited in a desiccator. During the vascular intervention of the framework 10, the surroundings of the marker element 30 also come into contact with blood. This exemplary embodiment therefore has the advantage that the pores of the abluminal top face of the marker element 30 with the porous layer 35, on account of the adhesion peptides attached by means of APTES, absorb blood constituents that lead to an improved adhesion and healing. It is thus ensured that the marker grows in quickly and remains reliably ingrown in the vessel tissue even after degradation of the framework 10 and the polymer active substance layer.

In a further alternative exemplary embodiment the hollow cylinder 40 with the portions 31, following the plasma electrolytic treatment, is rinsed multiple times in demineralised water following removal of the balloon and is then additionally immersed in a solution containing the crosslinker APTES (aminopropyltriethoxysilane). By applying a negative pressure in a desiccator, the pores are saturated with the solution. Here, the reaction on the marker surface can also be thermally activated. There is then additionally a reaction with proteins, such as growth factors (for example VEGF) or antibodies, which have a particular affinity to the cells of the vessel wall. These bind covalently to the surface activated with APTES. The hollow cylinder 40 is then rinsed/dried and deposited in a desiccator. In this exemplary embodiment as well, the immobilised proteins cause the marker element 30 to become quickly ingrown into the vessel tissue and to remain in the tissue even after degradation of the framework 10 and the polymer active substance arranged thereon.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. A method for producing a marker element for an implant, the method comprising:
providing a planar or hollow body-shaped semi-finished product consisting of a material of the marker element;
cutting out at least one portion from the semi-finished product, in such a way that the portion remains connected via at least one web to a remaining material of the semi-finished product;
with the portion still connected to the semi-finished product via the at least one web:
covering a first surface of the portion; and
subsequently performing plasma electrolytic treatment of a second surface of the portion that is different from the first surface, and thereby forming a porous layer at least on the second surface of the portion by the plasma electrolytic treatment; and
separating the portion by severing the at least one web, wherein the separated portion of the semi-finished product forms the marker element.

2. The method according to claim 1, which comprises performing the plasma electrolytic treatment at a maximum bath voltage of more than 180 V with a pulsed voltage source that is electrically conductively connected to the semi-finished product.

3. The method according to claim 1, which comprises forming porous layer also at least in part on a side face of the cut-out portion transverse to the second surface by way of the plasma electrolytic treatment.

4. The method according to claim 1, which comprises subjecting the semi-finished product, in a region of the cut-out portion, to a pickling treatment with an acid prior to covering the first surface of the portion.

5. The method according to claim 1, which comprises, after the plasma electrolytic treatment, firstly immersing the semi-finished product, in a region of the at least one cut-out portion, in a solution comprising a crosslinker and subsequently immersing in a mixture comprising at least one material selected from the group consisting of proteins, further growth factors, enzymes, antibodies and peptides.

6. The method according to claim 1, which comprises immobilizing an organic compound or an inorganic compound capable of elution in a pore structure of the porous layer.

7. A method for producing a framework with a marker element for an implant, the method comprising:
producing the marker element by the method according to claim 1;
producing a framework for supporting the marker element;
introducing the separated portion of the semi-finished product forming the marker element into an opening of the framework and gluing the marker element into the opening.

8. The method according to claim 7, wherein the gluing step comprises applying a polymer solution to the marker element separated from the semi-finished product, applying the polymer solution by pressing in and/or filling the gap between the marker element and an opposite edge of the opening of the framework, and subsequently curing the polymer solution.

9. The method according to claim 7, which comprises providing the framework, at least in a predefined region, with a coating containing a pharmaceutically active substance, prior to the step of gluing the marker element.

10. The method according to claim 7, which comprises providing the framework, at least in a predefined region, with a coating containing a pharmaceutically active substance, after to the step of gluing the marker element.

11. A disc-shaped marker element for an implant, the marker element comprising:
a bottom face forming a first surface;
a top face forming a second surface; and
at least one side face connecting said bottom face and said top face;
said top face forming an abluminal face once the marker element has been secured to a framework of the implant; and
a porous layer generated by plasma electrolytic treatment covering said top face at least in part.

12. The marker element according to claim 11, wherein said at least one side face is also covered by said porous layer.

13. The marker element according to claim 11, wherein said porous layer is formed with pores having a mean pore diameter between 0.1 μm and 2 μm and/or a layer thickness between 0.3 μm and 10 μm.

14. The marker element according to claim 13, wherein said porous layer has a layer thickness between 0.5 μm and 4 μm.

15. The marker element according to claim 11, wherein said bottom face is formed with a groove-shaped pickling structure.

16. A framework for an implant, comprising a marker element according to claim 11, said marker element being glued into an opening of the framework and a top face of said marker element forming an abluminal face.

17. An implant, comprising a framework formed with an opening, and a marker element according to claim 14 glued into said opening of said framework, with a top face of said marker element forming an abluminal face of the implant.
